(19) United States
(12) Patent Application Publication
(10) Pub. No.: US 2025/0241741 A1
(43) Pub. Date: Jul. 31, 2025

(54) CIRCUMFERENTIAL ENDOANCHORS

(71) Applicant: Medtronic Vascular, Inc., Santa Rosa, CA (US)

(72) Inventors: Matthew P. NORGROVE, Galway (IE); Ciaran M. McGUINNESS, Galway (IE)

(21) Appl. No.: 19/016,957

(22) Filed: Jan. 10, 2025

Related U.S. Application Data
(60) Provisional application No. 63/625,330, filed on Jan. 26, 2024.

(51) Int. Cl. A61F 2/07

(52) U.S. Cl. CPC A61F 2/07; A61F 2210/0014; A61F 2220/0008; A61F 2230/0091

(57) ABSTRACT

An endoanchor for anchoring an endograft to an aortic wall. The endoanchor comprises a first end, a second end, and a body helically extending between the first end and the second end. The endoanchor has a deployed state in which the endoanchor is configured to apply a radial force around the circumference of the endograft to anchor the endograft to the aortic wall.

### CROSS-REFERENCE TO RELATED APPLICATION

This application claims the benefit of U.S. provisional application Ser. No. 63/625,330 filed Jan. 26, 2024, the disclosure of which is hereby incorporated in its entirety by reference.

### TECHNICAL FIELD

The present disclosure relates to circumferential endoanchors and endovascular delivery systems for delivering endograft circumferential endoanchors.

### BACKGROUND

An aortic aneurysm may be an enlarged area in an aorta (e.g., an abdominal aorta). The aortic aneurysm may form from the degradation of elastin and/or interstitial collagen, which may alter the structural integrity of the aortic wall, which may weaken it. One current treatment of an aortic aneurysm may be endovascular surgery which utilizes an implant (e.g., stent graft). Another more invasive option is open surgery.

Endovascular procedures are minimally invasive techniques to deliver clinical treatments in a patient's vasculature (e.g., treatment of aortic aneurysms). One example of a clinical treatment used in an endovascular procedure is deployment of a stent graft. A conventional stent graft typically includes a radially expandable reinforcement structure, e.g., formed from a plurality of annular stent rings, and a cylindrically shaped layer of graft material defining a lumen to which the stent rings are coupled. The stent graft is placed inside a patient's vasculature (e.g., blood vessel) to bridge a diseased blood vessel segment (e.g., an aneurismal, dissected, or torn blood vessel segment), and thereby excluding hemodynamic pressures of blood flow from the diseased blood vessel segment.

The proximal end of the stent graft is expanded onto a landing zone of the diseased blood vessel. The anatomy of the landing zone may compromise the fixation and sealing of the stent graft, thereby potentially causing leaking and/or movement of the stent graft. Endoanchors may be applied to the proximal end of the stent graft to anchor the stent graft by penetrating the stent graft and blood vessel wall.

### SUMMARY

In an embodiment, an endoanchor for anchoring an endograft to an aortic wall is disclosed. The endoanchor includes a first end, a second end, and a body helically extending between the first end and the second end. The endoanchor has a deployed state in which the endoanchor is configured to apply a radial force around a circumference of the endograft to anchor the endograft to the aortic wall.

The endoanchor may have a pre-set state in which the endoanchor has a nominal pre-set diameter less than a nominal deployed diameter in the deployed state of the endoanchor. The nominal pre-set diameter may be less than the nominal deployed diameter by 5% or less. The body may be formed of a shape memory material to set the nominal pre-set diameter. The body may helically extend to form a circular helix shape. The body may have a body longitudinal axis and the endograft may have an endograft longitudinal axis. The endoanchor in the deployed state may be configured to align the body longitudinal axis and the endograft longitudinal axis. The body may include one or more helical turns (e.g., 1 to 5 helical turns). One of the one or more helical turns may have a pitch and the body may have a body radius. The endoanchor may have a ratio of the pitch to the body radius of 1:2 to 3:1. The body may have a body longitudinal height and endograft may have an endograft longitudinal height. The body longitudinal height may correspond to the endograft longitudinal height.

In another embodiment, an endoanchor delivery system is disclosed. The system may include a handle system, an applier catheter, and an endoanchor. The applier catheter extends from the handle system and defines a lumen therein. The applier catheter has a proximal portion and a distal portion. The endoanchor having a first end, a second end, and a body helically extending between the first end and the second end. The endoanchor has a delivery state within the applier catheter in which the endoanchor has an uncoiled shape. The endoanchor has a deployed state in which the endoanchor is configured to apply a radial force around a circumference of an endograft to anchor the endograft to an aortic wall. The applier catheter is configured to deploy the endoanchor from the distal portion to transition the endoanchor from the delivery state to the deployed state.

The endoanchor in the delivery state may be constrained by the applier catheter. At least a portion of the endoanchor may occupy the distal portion of the applier catheter in the delivery state. The endoanchor hay have a leading tip formed of a conductive material configured to activate to contact an end portion of the endograft when the endoanchor is being deployed. A remaining portion of the endoanchor excluding the leading tip may be insulated.

In yet another embodiment, a method of deploying an endoanchor to anchor an endograft to an aortic wall of an aorta is disclosed. The method includes deploying the endograft within the aorta by landing the endograft at a landing zone of the aortic wall. The method further includes delivering the endoanchor in a delivery state within the endograft to the landing zone of the aortic wall. The method further includes deploying the endoanchor within the landing zone to apply a radial force around a circumference of the endograft to anchor the endograft to the aortic wall.

The deploying step may include applying the radial force without the endoanchor directly contacting the aortic wall. The deploying step may include releasing the endoanchor in the delivery state from an endoanchor applier. The releasing step includes axially translating the endoanchor applier while releasing the endoanchor. The releasing step may include radially rotating the endoanchor applier while releasing the endoanchor.

### BRIEF DESCRIPTION OF THE DRAWINGS

FIG. 1A is a perspective view of a prior art endoanchor delivery system including an endoanchor guide and an endoanchor applier.

FIG. 1B is a perspective view of a prior art endoanchor cassette defining apertures for receiving endoanchors.

FIG. 1C is a cross section view of an abdominal aorta and a side view of prior art endoanchor catheter in a deployment state deploying an endoanchor to anchor a stent graft to a wall of the abdominal aorta.

FIG. 1D is a magnified view of a distal portion of the endoanchor guide in a deployment state deploying the endoanchor shown in FIG. 1C.

FIG. 2A is a schematic, perspective view of an endograft having first endoanchor deployed in a proximal section of the endograft and a second endoanchor deployed in a distal section of the endograft.

FIG. 2B is a cross-sectional view of the endograft and the second endoanchor taken about line 2B-2B of FIG. 2A.

FIG. 2C is a cross-sectional view of the endograft and the first and second endoanchors taken about line 2C-2C of FIG. 2A.

FIG. 2D is a cross-sectional view of an abdominal aorta with a deployed endograft and a deployed endoanchor partially deployed through an endoanchor applier.

FIG. 2E is a cross-sectional side view of a distal region of the endoanchor applier of FIG. 2D.

FIG. 3A depicts a cross-sectional view of an abdominal aorta with a deployed endograft and a deployed endoanchor through an endoanchor applier.

FIG. 3B depicts a schematic, perspective view of an endograft having an endoanchor extending within the endograft.

FIG. 3C depicts an exploded view of region 4C shown in FIG. 4B showing a conductive leading tip of the endoanchor.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

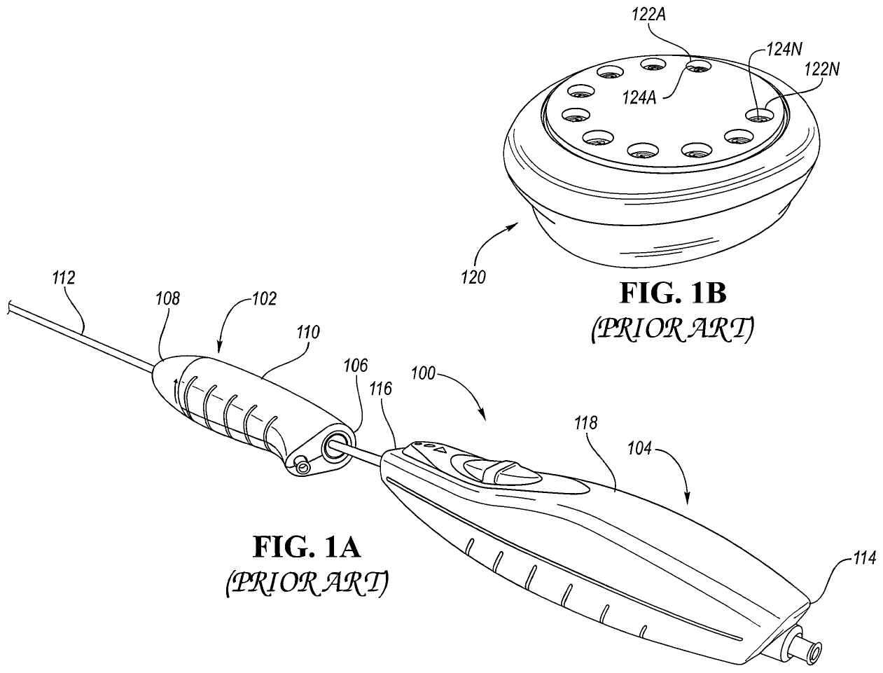

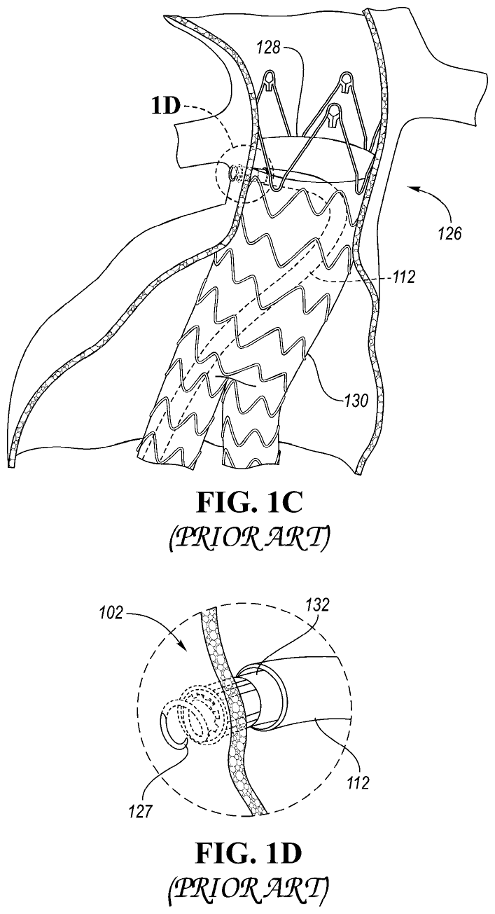

FIG. 1A is a perspective view of prior art endoanchor delivery system 100 including endoanchor guide 102 and endoanchor applier 104. Endoanchor guide 102 includes proximal end 106 and distal end 108 and body 110 extending therebetween. Endoanchor sheath 112 extends from distal end 108 of endoanchor guide 102. Endoanchor applier 104 include proximal end 114 and distal end 116 and body 118 extending therebetween.

FIG. 1B is a perspective view of prior art endoanchor cassette 120 defining apertures 122A through 122N for receiving endoanchors 124A through 124N, respectively. One of endoanchors 124A through 124N is loaded into a distal end of endoanchor applier 104. Endoanchor delivery system 100 is configured to deliver the loaded endoanchor to a delivery site to provide fixation and sealing between an endovascular implant (e.g., an endovascular stent graft) and a wall of native blood vessel (e.g., a thoracic aorta or an abdominal aorta). Endoanchor applier 104 is withdrawn from the vasculature of a patient after deployment of the loaded endoanchor so that a subsequent endoanchor may be loaded into the distal end of endoanchor applier 104.

FIG. 1C is a cross section view of abdominal aorta 126 and a side view of endoanchor applier 104 in a deployment state deploying loaded prior art endoanchor 127 to anchor proximal end 128 of stent graft 130 to a wall of abdominal aorta 126. FIG. 1D is a magnified view of a distal portion of prior art endoanchor applier 104 in a deployment state deploying loaded endoanchor 127. The distal portion of endoanchor guide 102 includes endoanchor sheath 112 and endoanchor applier 132. As shown in FIG. 1C, endoanchor applier 132 is curved by deflectable endoanchor sheath 112 such that endoanchor applier 132 and endoanchor 127 are apposed against stent graft 130. Distal portion of endoanchor applier 132 is about 90 degrees relative to stent graft 130 such that endoanchor applier 132 reaches stent graft 130.

Challenging anatomy may make deployment of endoanchors difficult. The acute angulation in the abdominal aorta and/or the overall aorta size may make proper deflection and orientation of the staple application system difficult. This positioning of the applicator may be relatively time consuming, adding to the procedural length to deploy the endoanchor. Moreover, the endoanchors may be deployed through both a stent graft and an aortic wall, thereby adding to the force to deploy the endoanchor.

In light of the foregoing, what is needed is an endoanchor delivery system in which one or more endoanchors may be deployed without positioning an applicator against an endograft and/or deploying one or more endoanchors without piercing an endograft and/or an aortic wall.

In one or more embodiments, endoanchor delivery systems for deploying circumferential endoanchors are disclosed. The endoanchor delivery system includes an endoanchor catheter. In one or more embodiments, the endoanchor applier is configured to deploy one or more circumferential endoanchor(s). More than one circumferential endoanchor may be deployed successively during one procedure. The endoanchor applier may apply the circumferential endoanchor(s) without positioning the endoanchor against the endograft. In one or more embodiments, the circumferential endoanchor is deployed without piercing the endograft and/or aortic wall.

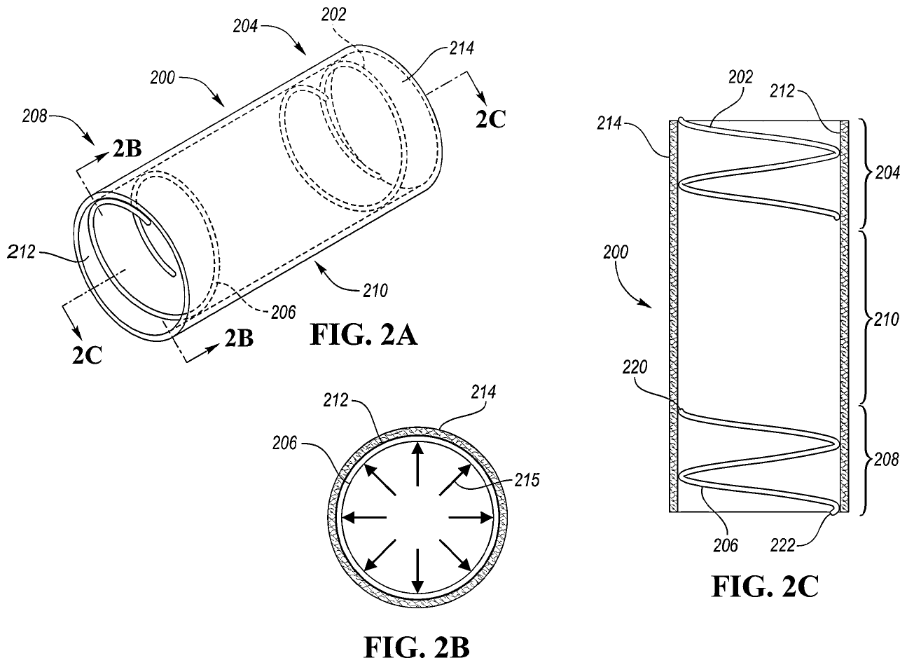

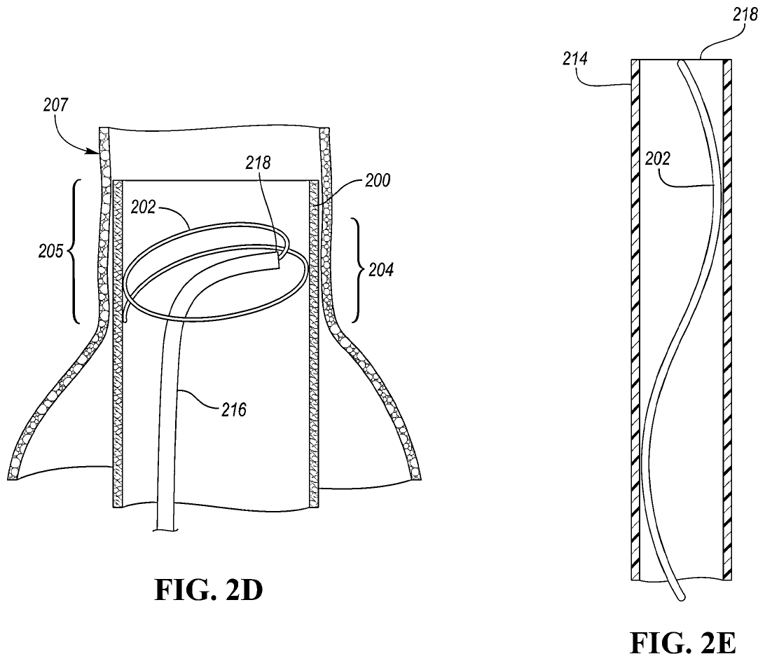

FIG. 2A is a schematic, perspective view of endograft 200 having first endoanchor 202 deployed in proximal section 204 of endograft 200 and second endoanchor 206 deployed in distal section 208 of endograft 200. FIG. 2B is a cross-sectional view of endograft 200 and second endoanchor 206 taken about line 2B-2B of FIG. 2A. FIG. 2C is a cross-sectional view of endograft 200, first endoanchor 202, and second endoanchor 206 taken about line 2C-2C of FIG. 2A.

As shown in FIG. 2A, endograft 200 is in a deployed position. Endograft 200 may be deployed in an ascending aorta, aortic arch, thoracic aorta, or an abdominal aorta of a patient or in any peripheral vessel thereof (e.g., renal artery, iliac artery, etc.). Medial section 210 may span an aneurysm in the blood vessel (e.g., abdominal aorta). As shown in FIG. 2D, proximal section 204 of endograft 200 may land on proximal landing zone 205 of the wall of abdominal aorta 207. Distal section 208 of endograft 200 may land on a distal landing zone (not shown) of the abdominal aorta wall. The aneurysm may extend between proximal landing zone 205 and the distal landing zone. In other embodiments, only one endoanchor may be deployed (e.g., at the proximal end or the distal end). One or more endoanchors may also be deployed in a medial portion of the stent graft to provide improved seal adjacent to a fenestrated or branching portion.

Endograft 200 has inner surface 212 and outer surface 214 with a body extending therebetween. Endograft 200 may be any form of an endovascular implant (e.g., an endovascular stent graft) implantable against a wall of a native blood vessel (e.g., a thoracic aorta or an abdominal aorta). First endoanchor 202 and second endoanchor 206 are shown in a deployed state in FIG. 2A. In the deployed state, first endoanchor 202 and second endoanchor 206 are configured to apply a radial force (e.g., as shown by arrows 215 in FIG. 2B) within endograft 200 against inner surface 212. The radial force acts upon endograft 200 to anchor endograft 200 to an aortic wall of the patient at the landing zone(s), thereby resisting movement of endograft 200 out of its deployment position. The anchoring may also resist leakage between outer surface 214 of endograft 200 and the aortic wall, for example, by improving the seal between the stent graft and the vessel wall. In one or more embodiments, the endoanchor(s) are configured to anchor endograft 200 without puncturing or piercing endograft 200 or the aortic wall.

In one or more embodiments, the endoanchor(s) apply the radial force around a circumference (e.g., a portion of the entire circumference or the entire circumference) of endograft 200. The radial circumferential force may be applied through helically shaped endoanchors (e.g., first endoanchor 202 and second endoanchor 206 are helically shaped endoanchors). The endoanchor(s) may be formed of a shape memory material. The shape memory material may be a shape memory alloy such as Nitinol. The shape memory material may be used to form a pre-set shape in a pre-set state of the endoanchor.

The endoanchor may have a pre-set state, a delivery state, and a deployed state. The endoanchor may have the shape of a helix (e.g., circular helix) having a radius (e.g., a nominal radius) in its pre-set state and its deployed state. In the pre-set state, the nominal radius of the endoanchor may be slightly greater than the nominal radius of inner surface 212 of endograft 200 in a deployed state. A slightly greater radius may be any of the following percentages or in a range of any two of the following percentages: 0.25%, 0.5%, 0.75%, 1%, 2%, 3%, 4%, or 5%, or a higher percentage depending on the material, strength and anatomy. The slightly greater pre-set state is configured to provide radial force (e.g., constant radial force) against endograft 200 and the aortic wall in the deployed state.

The delivery state may correspond to a constrained shape (e.g., the shape shown in FIG. 2E with first endoanchor 202 constrained within endoanchor applier 216). The endoanchor may be pulled into an uncoiled shape (e.g., a straight shape) or an elongated, less coiled shape for loading into endoanchor applier 216. The endoanchor may be loaded in a cold bath to lower the loading force (e.g., lowering the tendency for the endoanchor to revert to its preset shape). The temperature of the cold bath may be any of the following temperatures or in a range of any two of the following temperatures: 33, 34, 35, 40, 45, and 50° F. As the endoanchor is deployed (e.g., advanced through distal end 218 of endoanchor applier 216 and/or endoanchor applier 216 is retracted to release the endoanchor), the elevated temperature within the patient's body helps the endoanchor take on its pre-set shape.

The helical shape characteristic may include regular space curves with tangent lines at a constant angle to a fixed axis. Regular may refer to the space curves with no or slight deviations from the overall shape of the helix. The percentage of slight deviations from the longitudinal axis may be any of the following percentages or in a range of any two of the following percentages: 0.1%, 0.5%, 1%, 2%, 3%, 4%, and 5%. The fixed axis may be the longitudinal axis of the endoanchor in the pre-set state and/or deployed state. The helical shape characteristic may be a circular helix shape having a constant or nominal radius, curvature, and/or torsion. In the deployed state, the endoanchor longitudinal axis may be aligned with the endograft longitudinal axis to increase (e.g., maximize) the radial force exerted on the endograft and the aortic wall.

As shown in FIG. 2C, each of first endoanchor 202 and second endoanchor 206 includes 1.5 complete helical turns. A complete helical turn may refer to the helix extending 360 degrees around the entire circumference of endograft 200. The endoanchor may include any of number of the following helical turns or in a range of any two of the following number of helical turns: 1, 1.5, 2, 2.5, 3, 3.5, 4, 4.5, and 5. The number of helical turns may be selected depending on the amount of force intended to be imparted on the endograft and/or the aortic wall and the axial length of the landing zone(s). The pitch of a complete helical turn may refer to the height of the helical turn parallel to the longitudinal axis of the endoanchor. In the deployed state, the ratio of the pitch to the radius of the endoanchor may be selected to determine the number of helical turns. The ratio may be any of the following values or in a range of any two of the following values: 1:2, 1:1, 3:2, 2:1, 5:2, and 3:1, or a higher ratio. In one or more embodiments, the two turns may overlap each other to increase radial force at different portions of the endograft and/or aortic wall. The first two turns of the coil may overlap, and the remaining revolutions may have a ratio. This construction may help with scaling.

The endoanchor has an endoanchor height measured along the longitudinal axis of the endoanchor between the first and second ends of the endoanchor (e.g., first end 220 and second end 222 of second endoanchor 206) when the endoanchor is in the deployed state. The landing zone of the endograft has a landing zone height measured along the longitudinal axis of the endograft. In one or more embodiments, the endoanchor height corresponds to the landing zone height to apply a radial force to the endograft in the landing zone. In one or more embodiments, the endoanchor height is substantially equal to or equal to the landing zone height such that the radial force from the endoanchor is applied to the endograft through substantially all or all the landing zone height. In such embodiments, the endoanchor may only contact the endograft in the landing zone and the endoanchor does not directly contact the aortic wall, thereby reducing potential trauma to the aortic wall. In other embodiments, the endoanchor height is less than the landing zone height such that the radial force is applied to less than the entire height of the landing zone. These embodiments may be applied when the radial force is selectively applied to a portion of the landing zone based on the anatomy within the aortic region of the patient. In some embodiments, the endoanchor(s) may extend beyond the edge of the graft material such that a portion of the endoanchor contacts the vessel wall and another portion contacts the inner surface of the stent graft. For example, if the endoanchor is at the proximal edge of the stent graft, the endoanchor may extend proximally to the proximal edge and if at the distal edge it may extend distally to the distal edge. Such an embodiment may be beneficial if the edge of the stent graft is adjacent or abuts a branch vessel that a physician does not wish to block. The endoanchor may extend across the ostium of the branch vessel without blocking blood flow thereto and the extended length of the endoanchor may provide increased migration resistance to the endoanchor and the stent graft. An endoanchor extending across the edge of the graft material may also promote scaling of the graft material to the vessel wall.

As shown in FIG. 2D, first endoanchor 202 is being deployed from distal end 218 of endoanchor applier 216 whereby first endoanchor 202 is in a partially deployed state. An actuator may be configured to control the position of a plunger towards distal end 218 of endoanchor applier 216. Non-limiting examples of actuators include an electro-mechanical handle or one or more buttons on a handle control. The plunger is configured to engage or contact a proximal end of the endoanchor to push the endoanchor of the tip of endoanchor applier 216. In one or more embodiments, a retractable sheath may be utilized to deploy the endoanchor. A retractable outer sheath may be manually retracted at a proximal end of endoanchor applier 216. As the sheath is retracted, the endoanchor expands to engage the inner lumen of the graft. Sutures may be attached to a proximal end of the endoanchor to recapture and/or cut the endoanchor for a final deployment. As first endoanchor 202 is being deployed, endoanchor applier 216 may be advanced distally such that additional portion of first endoanchor 202 expand into and contact additional regions of inner surface 212 of endograft 200. Distal end 218 is spaced apart from inner surface 212 of endograft 200 as first endoanchor 202 is being deployed.

As further shown in FIG. 2D, distal end 218 of endoanchor applier 216 is oriented at about 90 degrees from endograft 200 as first endoanchor 202 is being released from endoanchor applier 216. In other embodiments, endoanchor applier 216 may be in other orientations (e.g., in a vertical/straight position parallel to its longitudinal axis and/or the longitudinal axis of the vessel) as first endoanchor 202 is being deployed as first endoanchor 202 expands to the pre-set state and orients in an aligned position (e.g., the longitudinal axes of the endoanchor and endograft align) independent of the orientation of endoanchor applier 216.

As shown in FIG. 2A, first end 220 and second end 222 of second endoanchor 206 do not include any sharp edges. First end 220 and second end 222 may have smooth surfaces and edges to resist second endoanchor 206 from piercing the aortic wall or an endograft. In these embodiments, the endoanchor does not connect to the endograft (other than contacting the inner surface of the endograft) or puncture the aortic wall. The cross-sectional shape of the endoanchor may be curved or circular to provide a smooth surface along the length of the body of the endoanchor. Any attribute or characteristic set forth above with respect to second or first endoanchor may also apply to the other endoanchors, such as the smooth surface and edges.

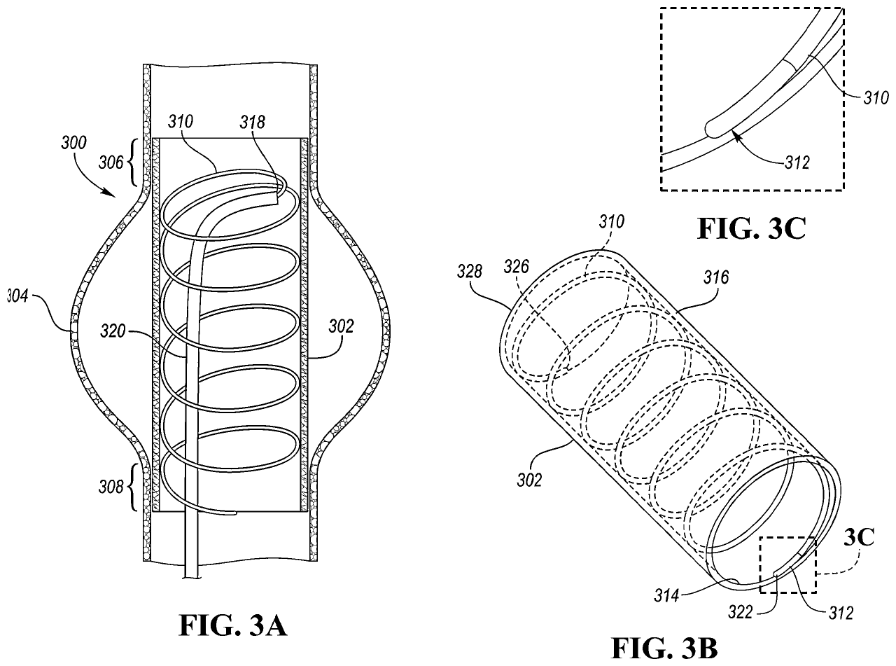

FIG. 3A depicts a cross-sectional view of abdominal aorta 300 having endograft 302 deployed therein across aneurysm 304. Endograft 302 lands on proximal landing zone 306 and distal landing zone 308. FIG. 3B depicts a schematic, perspective view of endograft 302 having endoanchor 310, in a deployed state, extending the entire longitudinal length of endograft 302. FIG. 3C depicts an exploded view of region 3C shown in FIG. 3B showing conductive tip 312 of endoanchor 310.

Endograft 302 has inner surface 314 and outer surface 316 with a body extending therebetween. In the deployed state, endoanchor 310 is configured to apply a radial force within endograft 302 against inner surface 314 along the entire longitudinal length of endograft 302. In other embodiments, endoanchor 310 may only extend a portion of the longitudinal length of endograft 302 provided that endoanchor 310 extends an axial portion of proximal landing zone 306 and distal landing zone 308. The radial force acts upon endograft 302 to anchor endograft 302 to an aortic wall of patient at the landing zones, thereby resisting movement of endograft 302 out of its deployment position. The anchoring may also resist leakage/improve seal between outer surface 316 of endograft 302 and the aortic wall. In one or more embodiments, endoanchor 310 is configured to anchor endograft 302 without puncturing or piercing endograft 302 or the aortic wall.

In one or more embodiments, endoanchor 310 applies a radial force around the entire circumference of endograft 302. Endoanchor 310 may have a pre-set state, a delivery state, and a deployed state. Endoanchor 310 may have the shape of a helix (e.g., circular helix) having a radius (e.g., a nominal radius) in its pre-set state and deployed state. In the pre-set state, the nominal radius of endoanchor 310 may be slightly greater than the nominal radius of inner surface 314 of endograft 302. The slightly greater pre-set state is configured to spring outward upon deployment to provide the radial force against endograft 302 and the aortic wall in the deployed state. The delivery state of endoanchor 310 may share one or more attributes with the delivery state of endoanchor 206. The helical shape characteristic of endoanchor 310 may share one or more attributes with the helical shape characteristic of endoanchor 206.

As shown in FIG. 3B, endoanchor 310 includes 7 complete helical turns. Endoanchor 310 may include less or more helical turns (e.g., 4, 5, 6, 8, 9, 10, or a range between two of these values) depending on the amount of force intended to be imparted on the endograft and/or the aortic wall and the axial length of the landing zone(s). The ratio of the pitch to the radius of endoanchor 310 may be selected from the values/ranges identified above.

As shown in FIG. 3A, endoanchor 310 is being deployed from distal end 318 of endoanchor applier 320 whereby endoanchor 310 is in a partially deployed state. As endoanchor 310 is being deployed (e.g., by being pushed out of applier), endoanchor applier 320 may be advanced distally such that additional portions of endoanchor 310 expand into and contact additional regions of inner surface 314 of endograft 302. Alternatively, endoanchor 310 may be deployed from proximal landing zone 306 to distal landing zone 308 by retracting endoanchor applier 320 in a proximal direction as the endoanchor is pushed out of the applier or the applier is retracted relative to the endoanchor. As endoanchor applier 320 is being advanced/retracted, it may be rotated to mimic the contour of the helical shape of endoanchor 310 as it is being deployed. As further shown in FIG. 3A, distal end 318 of endoanchor applier 320 may be oriented at about 90 degrees and spaced apart from endograft 302 as endoanchor 310 is being released from endoanchor applier 320. In other embodiments, endoanchor applier 320 may be in other orientations (e.g., in a vertical/straight position parallel to its longitudinal axis and/or the longitudinal axis of the vessel) as endoanchor 310 is being deployed as endoanchor 310 expands to the pre-set state and orients in an aligned position (e.g., the longitudinal axes of the endoanchor and endograft align) independent of the orientation of endoanchor applier 320.

As shown in FIGS. 3B and 3C, leading tip 322 (e.g., the tip that exits endoanchor applier 320 first) of endoanchor 310 may be formed of a conductive material configured to be activated outside the patient by a clinician. The rest of endoanchor 310 may be formed of an insulating material (or a conductive material that is insulated with an insulating coating) such that conductive activation only activates leading tip 322. Leading tip 322 may be activated (e.g., heated) to improve initial contact (e.g., anchoring) with distal end 324 of endograft 302 during deployment. Trailing tip 326 may also include the conductive material for improving contact with proximal end 328 of endograft 302. The heat may allow the tips of the endoanchor to pierce the graft material, thereby aiding in the fixation of the endoanchor to the graft over the lifetime of use. In such an embodiment, the tips of the endoanchor do not pierce the vessel wall while piercing the graft material.

In one or more embodiments, the endoanchors (e.g., endoanchors 202, 206, and/or 310) may include a radiopaque marking material (e.g., gold, platinum, iridium, etc.) configured to be visualized under fluoroscopy. The use of a radiopaque marking material is configured to aid the clinician in deploying each endoanchor in an appropriate location. In at least one embodiment, the endoanchor(s) may have one or more radiopaque markers at their tip or leading edge (e.g., tip of a helical anchor) to assist with visualization of the endoanchor as it is being positioned and/or inserted. In another embodiment, the endoanchor(s) may alternatively or additionally have one or more radiopaque markers on their base or the trailing edge to assist with visualization of endoanchor during and/or after deployment. The radiopaque markers may have a shape that is asymmetrical on at least one axis in a plane visible under fluoroscopy to assist with orientation. For example, a letter “E” or letter “C” may be used, as this allows the physician to know whether the marker is facing forwards or backwards.

The endoanchor of one or more embodiments may be preloaded into a catheter before delivery to a laboratory or hospital. In one or more embodiments, two or more endoanchors may be loaded into a single catheter so that repeat loads or multiple insertions may be avoided.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An endoanchor for anchoring an endograft to an aortic wall, the endoanchor comprising:
a first end;
a second end; and
a body helically extending between the first end and the second end, the endoanchor has a deployed state in which the endoanchor is configured to apply a radial force around a circumference of the endograft to anchor the endograft to the aortic wall.

2. The endoanchor of claim 1, wherein the endoanchor has a pre-set state in which the endoanchor has a nominal pre-set diameter greater than a nominal deployed diameter of the endograft.

3. The endoanchor of claim 2, wherein the nominal pre-set diameter is greater than the nominal deployed diameter by 5% or less.

4. The endoanchor of claim 2, wherein the body is formed of a shape memory material to set the nominal pre-set diameter.

5. The endoanchor of claim 1, wherein the body helically extends to form a circular helix shape.

6. The endoanchor of claim 1, wherein the body has a body longitudinal axis, the endograft has an endograft longitudinal axis, and the endoanchor in the deployed state is configured to align the body longitudinal axis and the endograft longitudinal axis.

7. The endoanchor of claim 1, wherein the body includes one or more helical turns.

8. The endoanchor of claim 7, wherein the body includes 1 to 5 helical turns.

9. The endoanchor of claim 1, wherein one of the one or more helical turns has a pitch, the body has a body radius, and the endoanchor has a ratio of the pitch to the body radius of 1:2 to 3:1.

10. The endoanchor of claim 1, wherein the body has a body longitudinal height, the endograft has an endograft longitudinal height, and the body longitudinal height corresponds to the endograft longitudinal height.

11. An endoanchor delivery system comprising:
a handle system;
an applier catheter extending from the handle system and defining a lumen therein, the applier catheter having a proximal portion and a distal portion; and
an endoanchor having a first end, a second end, and a body helically extending between the first end and the second end, the endoanchor has a delivery state within the applier catheter in which the endoanchor has an uncoiled shape, the endoanchor has a deployed state in which the endoanchor is configured to apply a radial force around a circumference of an endograft to anchor the endograft to an aortic wall, and the applier catheter is configured to deploy the endoanchor from the distal portion to transition the endoanchor from the delivery state to the deployed state.

12. The endoanchor delivery system of claim 11, wherein the endoanchor in the delivery state is constrained by the applier catheter.

13. The endoanchor delivery system of claim 11, wherein at least a portion of the endoanchor occupies the distal portion of the applier catheter in the delivery state.

14. The endoanchor delivery system of claim 11, wherein the endoanchor has a leading tip formed of a conductive material configured to activate to contact an end portion of the endograft when the endoanchor is being deployed.

15. The endoanchor delivery system of claim 14, wherein a remaining portion of the endoanchor excluding the leading tip is insulated.

16. A method of deploying an endoanchor to anchor an endograft to an aortic wall of an aorta, the method comprising:
deploying the endograft within the aorta by landing the endograft at a landing zone of the aortic wall;
delivering the endoanchor in a delivery state within the endograft to the landing zone of the aortic wall; and
deploying the endoanchor within the landing zone to apply a radial force around a circumference of the endograft to anchor the endograft to the aortic wall.

17. The method of claim 16, wherein the deploying step includes applying the radial force without the endoanchor directly contacting the aortic wall.

18. The method of claim 16, wherein the deploying step includes releasing the endoanchor in the delivery state from an endoanchor applier.

19. The method of claim 18, wherein the releasing step includes axially translating the endoanchor applier while releasing the endoanchor.

20. The method of claim 19, wherein the releasing step includes radially rotating the endoanchor applier while releasing the endoanchor.
